(19) United States
(12) Patent Application Publication
(10) Pub. No.: US 2006/0121110 A1
(43) Pub. Date: Jun. 8, 2006

(54) Formulations of substituted benzoxazoles

(75) Inventors: James A. Provost, Waltham Chase (GB); Trevor I. Armstrong, Hampshire (GB); Zerina B. Shafi, Fareham (GB); Marc S. Tesconi, Monroe, NY (US); Mannching Sherry Ku, Thiells, NY (US)

Correspondence Address: COZEN O' CONNOR, P. C., 1900 MARKET STREET, PHILADELPHIA, PA, 19103-3508, US

(73) Assignee: Wyeth, Madison, NJ (US)

(21) Appl. No.: 11/290,197

(22) Filed: Nov. 30, 2005

Related U.S. Application Data
(60) Provisional application No. 60/632,375, filed on Dec. 2, 2004.

(51) Int. Cl. A61K 31/428; A61K 31/423; A61K 31/4184; A61K 9/20

(52) U.S. Cl. 424/464; 514/367; 514/375; 514/394

(57) ABSTRACT

The present invention relates to solid dosage formulations that include ERβ-selective ligands that contain benzoxazole, and processes for manufacture of said formulations, more particularly to novel formulations and processes for manufacture of formulations containing the ERβ-selective ligand, ERB-041.

### CROSS-REFERENCE TO RELATED APPLICATION

This present invention claims benefit of priority from provisional U.S. Patent Application Ser. No. 60/632,375 filed Dec. 2, 2004, which is incorporated herein by reference in its entirety.

### FIELD OF THE INVENTION

The present invention relates to solid dosage formulations that include ERβ-selective ligands that contain benzoxazole (or benzothiazole or benzoimidazole), and processes for manufacture of said formulations, more particularly to novel formulations and processes for manufacture of formulations containing the ERβ-selective ligand, ERB-041.

### BACKGROUND OF THE INVENTION

This invention relates to formulations for substituted benzoxazoles (and benzothiazoles and benzodiazoles), which are useful as estrogenic agents.

The pleiotropic effects of estrogens in mammalian tissues have been well documented, and it is now appreciated that estrogens affect many organ systems [Mendelsohn and Karas, New England Journal of Medicine 340: 1801-1811 (1999), Epperson, et al., Psychosomatic Medicine 61: 676-697 (1999), Crandall, Journal of Women's Health & Gender Based Medicine 8: 1155-1166 (1999), Monk and Brodaty, Dementia & Geriatric Cognitive Disorders 11: 1-10 (2000), Hum and Macrae, Journal of Cerebral Blood Flow & Metabolism 20: 631-652 (2000), Calvin, Maturitas 34: 195-210 (2000), Finking, et al., Zeitschrift fur Kardiologie 89: 442-453 (2000), Brincat, Maturitas 35: 107-117 (2000), Al-Azzawi, Postgraduate Medical Journal 77: 292-304 (2001)]. Estrogens can exert effects on tissues in several ways, and the most well characterized mechanism of action is their interaction with estrogen receptors leading to alterations in gene transcription. Estrogen receptors are ligand-activated transcription factors and belong to the nuclear hormone receptor superfamily. Other members of this family include the progesterone, androgen, glucocorticoid and mineralocorticoid receptors. Upon binding ligand, these receptors dimerize and can activate gene transcription either by directly binding to specific sequences on DNA (known as response elements) or by interacting with other transcription factors (such as AP1), which in turn bind directly to specific DNA sequences [Moggs and Orphanides, EMBO Reports 2: 775-781 (2001), Hall, et al., Journal of Biological Chemistry 276: 36869-36872 (2001), McDonnell, Principles of Molecular Regulation 351-361 (2000)]. A class of “coregulatory” proteins can also interact with the ligand-bound receptor and further modulate its transcriptional activity [McKenna, et al., Endocrine Reviews 20: 321-344 (1999)]. It has also been shown that estrogen receptors can suppress NFκB-mediated transcription in both a ligand-dependent and independent manner [Quaedackers, et al., Endocrinology 142: 1156-1166 (2001), Bhat, et al., Journal of Steroid Biochemistry & Molecular Biology 67: 233-240 (1998), Pelzer, et al., Biochemical & Biophysical Research Communications 286: 1153-7 (2001)].

Estrogen receptors can also be activated by phosphorylation. This phosphorylation is mediated by growth factors such as EGF and causes changes in gene transcription in the absence of ligand [Moggs and Orphanides, EMBO Reports 2: 775-781 (2001), Hall, et al., Journal of Biological Chemistry 276: 36869-36872 (2001)].

A less well-characterized means by which estrogens can affect cells is through a so-called membrane receptor. The existence of such a receptor is controversial, but it has been well documented that estrogens can elicit very rapid non-genomic responses from cells. The molecular entity responsible for transducing these effects has not been definitively isolated, but there is evidence to suggest it is at least related to the nuclear forms of the estrogen receptors [Levin, Journal of Applied Physiology 91: 1860-1867 (2001), Levin, Trends in Endocrinology & Metabolism 10: 374-377 (1999)].

Two estrogen receptors have been discovered to date. The first estrogen receptor was cloned about 15 years ago and is now referred to as ERα [Green, et al., Nature 320: 134-9 (1986)]. The second form of the estrogen receptor was found comparatively recently and is called ERβ [Kuiper, et al., Proceedings of the National Academy of Sciences of the United States of America 93: 5925-5930 (1996)]. Early work on ERβ focused on defining its affinity for a variety of ligands and indeed, some differences with ERα were seen. The tissue distribution of ERβ has been well mapped in the rodent and it is not coincident with ERα. Tissues such as the mouse and rat uterus express predominantly ERα, whereas the mouse and rat lung express predominantly ERβ [Couse, et al., Endocrinology 138: 4613-4621 (1997), Kuiper, et al., Endocrinology 138: 863-870 (1997)]. Even within the same organ, the distribution of ERα and ERβ can be compartmentalized. For example, in the mouse ovary, ERβ is highly expressed in the granulosa cells and ERα is restricted to the thecal and stromal cells [Sar and Welsch, Endocrinology 140: 963-971 (1999), Fitzpatrick, et al., Endocrinology 140: 2581-2591 (1999)]. However, there are examples where the receptors are coexpressed and there is evidence from in vitro studies that ERα and ERβ can form heterodimers [Cowley, et al., Journal of Biological Chemistry 272: 19858-19862 (1997)].

A large number of compounds have been described that either mimic or block the activity of 17β-estradiol. Compounds having roughly the same biological effects as 17β-estradiol, the most potent endogenous estrogen, are referred to as “estrogen receptor agonists”. Those which, when given in combination with 17β-estradiol, block its effects are called “estrogen receptor antagonists”. In reality there is a continuum between estrogen receptor agonist and estrogen receptor antagonist activity and indeed some compounds behave as estrogen receptor agonists in some tissues and estrogen receptor antagonists in others. These compounds with mixed activity are called selective estrogen receptor modulators (SERMS) and are therapeutically useful agents (e.g. EVISTA®) [McDonnell, Journal of the Society for Gynecologic Investigation 7: S10-S15 (2000), Goldstein, et al., Human Reproduction Update 6: 212-224 (2000)]. The precise reason why the same compound can have cell-specific effects has not been elucidated, but the differences in receptor conformation and/or in the milieu of coregulatory proteins have been suggested.

It has been known for some time that estrogen receptors adopt different conformations when binding ligands. However, the consequence and subtlety of these changes has been only recently revealed. The three dimensional structures of ERα and ERβ have been solved by co-crystallization with various ligands and clearly show the repositioning of helix 12 in the presence of an estrogen receptor antagonist that sterically hinders the protein sequences required for receptor-coregulatory protein interaction [Pike, et al., EMBO 18: 4608-4618 (1999), Shiau, et al., Cell 95: 927-937 (1998)]. In addition, the technique of phage display has been used to identify peptides that interact with estrogen receptors in the presence of different ligands [Paige, et al., Proceedings of the National Academy of Sciences of the United States of America 96: 3999-4004 (1999)]. For example, a peptide was identified that distinguished between ERα bound to the full estrogen receptor agonists 17β-estradiol and diethylstilbesterol. A different peptide was shown to distinguish between clomiphene bound to ERα and ERβ. These data indicate that each ligand potentially places the receptor in a unique and unpredictable conformation that is likely to have distinct biological activities.

The preparation of exemplary ERβ selective ligands, including 2-(3-fluoro-4-hydroxyphenyl)-7-vinyl-1,3-benzoxazol-5-ol (ERB-041), is described in U S. Pat. No. 6,794,403, incorporated herein by reference in its entirety.

As mentioned above, estrogens affect a panoply of biological processes. In addition, where gender differences have been described (e.g., disease frequencies, responses to challenge, etc.), it is possible that the explanation involves the difference in estrogen levels between males and females.

Given the importance of these compounds as pharmaceutical agents, it can be seen that effective formulations for delivery of the compounds is of great import. This invention is directed to these, as well as other, important ends.

### SUMMARY OF THE INVENTION

In some embodiments, the present invention provides pharmaceutical formulations comprising a pharmaceutically effective amount of an active pharmacological agent and a carrier or excipient system, the carrier or excipient system comprising:

a) a filler/diluent component comprising from about 40% to about 90% by weight of the pharmaceutical formulation;

b) a surface modifying agent component comprising from about 0.4% to about 15% by weight of the pharmaceutical formulation;

c) a disintegrant component from about 0.01% to about 10% by weight of the pharmaceutical formulation;

d) optionally, a second filler/diluent component comprising up to about 20% by weight of the pharmaceutical formulation; and

e) a lubricant component comprising from about 0.01% to about 5% by weight of the pharmaceutical formulation;

wherein the active pharmacological agent has the Formula I: 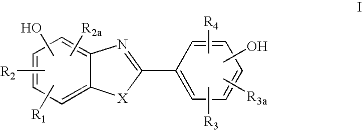
wherein

R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₅, R₆ are each, independently hydrogen, alkyl of 1-6 carbon atoms, or aryl of 6-10 carbon atoms;

X is O, S, or N R₇; and

R₇ is hydrogen, alkyl of 1-6 carbon atoms or aryl of 6-10 carbon atoms, —COR₅, —CO₂R₅ or —SO₂R₅;

or a pharmaceutically acceptable salt thereof.

In some embodiments, X is O. In some further embodiments, R₁ is alkenyl of 2-3 carbon atoms, which is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆. In some embodiments, the active ingredient is 2-(3-fluoro-4-hydroxyphenyl)-7-vinyl-1,3-benzoxazol-5-ol (ERB-041) or a pharmaceutically acceptable salt thereof.

The term halogen refers to chloro, bromo, fluoro or iodo, preferably fluoro. The alkyl of 1-6 carbon atoms (used alone or as part of a group e.g. alkoxy) may be a straight or branched alkyl e.g. methyl, ethyl, n-propyl, i-propyl or n-butyl. The cycloalkyl of 3-8 carbon atoms may be saturated or unsaturated and includes the moieties cyclopropyl, cyclobutyl, cyclopentyl and cyclohexyl. The trifluoroalkyl of 1-6 carbon atoms (used alone or as part of a group) may suitably be trifluoromethyl. Sulfoxoalkyl of 1-6 carbon atoms refers to the group —SO—R wherein R is an alkyl of 1-6 carbon atoms as defined above. Aryl of 6-10 carbon atoms refers to a mono or poly cyclic aromatic group. e.g., phenyl or napthyl. The 5 to 6 membered heterocyclic ring having 1 to 4 heteroatoms selected from O, N or S is a saturated, partially unsaturated or aromatic ring, e.g., a furanyl, pyranyl, pyridinyl, pyrimidinyl, pyrazinyl, morpholinyl, thiomorpholinyl, imidazolyl, oxazolyl, thioxazolyl, thienyl or piperidinyl ring. The alkynyl of 2-7 carbon atoms is a group having at least one triple bond, e.g., ethynyl. The alkenyl of 2-7 carbon atoms is a group having at least one double bond, e.g., vinyl. When the alkyl or alkenyl moieties are substituted they may be substituted with 1 or more substituents as defined above, e.g. by 1, 2 or 3 substituents which may be the same or different.

In some embodiments, the active pharmacological agent comprises from about 1.0% to about 50% by weight of the pharmaceutical formulation. In further embodiments, the active pharmacological agent comprises from about 1.0% to about 50% by weight of the pharmaceutical formulation; the filler/diluent component comprises from about 40% to about 90% by weight of the pharmaceutical formulation; the surface modifying agent component comprises from about 0.4% to about 15% by weight of the pharmaceutical formulation; the disintegrant component comprises from about 0.1% to about 10% by weight of the pharmaceutical formulation; the second filler/diluent component, when present, comprises up to about 20% by weight of the pharmaceutical formulation; and the lubricant component comprises from about 0.01% to about 5% by weight of the pharmaceutical formulation.

In some further embodiments, the active pharmacological agent comprises from about 1.5% to about 40% by weight of the pharmaceutical formulation; the filler/diluent component comprises from about 45% to about 85% by weight of the pharmaceutical formulation; the surface modifying agent component comprises from about 0.5 to about 12% by weight of the pharmaceutical formulation; the disintegrant component comprises from about 0.15% to about 8% by weight of the pharmaceutical formulation; the second filler/diluent component, when present, comprises from about 1% to about 20% by weight of the pharmaceutical formulation; and the lubricant component comprises from about 0.1% to about 2% by weight of the pharmaceutical formulation.

In still further embodiments, the active pharmacological agent comprises from about 2% to about 36% by weight of the pharmaceutical formulation; the filler/diluent component comprises from about 50% to about 85% by weight of the pharmaceutical formulation; the surface modifying agent component comprises from about 0.6 to about 10% by weight of the pharmaceutical formulation; the disintegrant component comprises from about 0.2% to about 6% by weight of the pharmaceutical formulation; the second filler/diluent component, when present, comprises from about 1.0% to about 20% by weight of the pharmaceutical formulation; and the lubricant component comprises from about 0.1% to about 1% by weight of the pharmaceutical formulation.

In some embodiments, the filler/diluent component comprises one or more of mannitol, lactose, sucrose, powdered cellulose, microcrystalline cellulose, maltodextrin, sorbitol, starch, xylitol or a metal aluminosilicate, for example magnesium aluminometasilicate (Neusilin®). In some further embodiments, the filler/diluent component comprises mannitol.

In some embodiments, the surface modifying agent component comprises a surfactant. In some embodiments, the surface modifying agent component comprises one or more of Poloxamer 188, sodium lauryl sulfate, polyoxyethylene sorbitan fatty acid ester, polyethylene glycol, polyoxyethylene castor oil derivative, docusate sodium, quaternary ammonium amine compound, sugar esters of fatty acid, or glycerides of fatty acid. In some further embodiments, the surface modifying agent component comprises one or more of Poloxamer 188 or sodium lauryl sulfate. In some further embodiments, the surface modifying agent component comprises Poloxamer 188.

In some embodiments, the disintegrant component comprises one or more of crosscarmellose sodium, pregelatinized starch, sodium starch glycolate, crospovidone, starch, alginic acid, sodium alginate, clay, cellulose floc, ion exchange resin, or effervescent systems based on food acids and an alkaline carbonate component. In some further embodiments, the disintegrant component comprises crosscarmellose sodium.

In some embodiments, the optional second filler/diluent component, when present, comprises one or more of microcrystalline cellulose, mannitol, lactose, sucrose, powdered cellulose, maltodextrin, sorbitol, starch, xylitol or a metal aluminosilicate, for example magnesium aluminometasilicate (Neusilin®). In some further embodiments, the optional second filler/diluent component, when present, comprises microcrystalline cellulose.

In some embodiments, the lubricant component comprises one or more of metallic stearate, fatty acid ester, fatty acid, fatty alcohol, glyceryl behenate, mineral oil, parrafin, hydrogenated vegetable oil, leucine, polyethylene glycol, metallic lauryl sulfate or sodium chloride. In some further embodiments, the metallic stearate is magnesium stearate, calcium stearate or zinc stearate. In still further embodiments, the lubricant component comprises magnesium stearate.

In some embodiments, the filler/diluent component comprises one or more of mannitol, lactose, sucrose, powdered cellulose, microcrystalline cellulose, maltodextrin, sorbitol, starch, xylitol, or a metal aluminosilicate; the surface modifying agent component comprises one or more of Poloxamer 188, sodium lauryl sulfate, polyoxyethylene sorbitan fatty acid ester, polyethylene glycol, polyoxyethylene castor oil derivative, docusate sodium, quaternary ammonium amine compound, sugar esters of fatty acid or glycerides of fatty acid; the disintegrant component comprises one or more of crosscarmellose sodium, pregelatinized starch, sodium starch glycolate, crospovidone, starch, alginic acid, sodium alginate, clay, cellulose floc, ion exchange resin, or effervescent systems based on food acids and an alkaline carbonate component; the disintegrant component comprises crosscarmellose sodium; the optional second filler/diluent component, when present, comprises one or more of microcrystalline cellulose, mannitol, lactose, sucrose, powdered cellulose, maltodextrin, sorbitol, starch, xylitol or a metal aluminosilicate, for example magnesium aluminometasilicate (Neusilin®); and the lubricant component comprises one or more of metallic stearate, fatty acid ester, fatty acid, fatty alcohol, glyceryl behenate, mineral oil, parrafin, hydrogenated vegetable oil, leucine, polyethylene glycol, metallic lauryl sulfate or sodium chloride.

In further embodiments, the filler/diluent component comprises one or more of mannitol, lactose, sucrose, powdered cellulose, microcrystalline cellulose, maltodextrin, sorbitol, starch, xylitol, or a metal aluminosilicate; the surface modifying agent component comprises one or more of Poloxamer 188, sodium lauryl sulfate, polyoxyethylene sorbitan fatty acid ester, polyethylene glycol, polyoxyethylene castor oil derivative, docusate sodium, quaternary ammonium amine compound, sugar esters of fatty acid or glycerides of fatty acid; the disintegrant component comprises one or more of crosscarmellose sodium, pregelatinized starch, sodium starch glycolate, crospovidone, starch, alginic acid, sodium alginate, clay, cellulose floc, ion exchange resin, or effervescent systems based on food acids and an alkaline carbonate component; the disintegrant component comprises crosscarmellose sodium; the optional second filler/diluent component, when present, comprises one or more of microcrystalline cellulose, mannitol, lactose, sucrose, powdered cellulose, maltodextrin, sorbitol, starch, xylitol or a metal aluminosilicate; and the lubricant component comprises one or more of metallic stearate, fatty acid ester, fatty acid, fatty alcohol, glyceryl behenate, mineral oil, parrafin, hydrogenated vegetable oils, leucine, polyethylene glycol, metallic lauryl sulfate or sodium chloride.

In some embodiments, the filler/diluent component comprises mannitol; the surface modifying agent component comprises Poloxamer 188; the disintegrant component comprises crosscarmellose sodium; the optional second filler/diluent component, when present, comprises microcrystalline cellulose; and the lubricant component comprises magnesium stearate.

In some embodiments, the active pharmacological agent comprises from about 1.0% to about 5% by weight of the pharmaceutical formulation; the filler/diluent component comprises from about 70% to about 90% by weight of the pharmaceutical formulation; the surface modifying agent component comprises from about 0.1% to about 2% by weight of the pharmaceutical formulation; the disintegrant component comprises from about 0.01% to about 2% by weight of the pharmaceutical formulation; the optional second filler/diluent component, when present, comprises from about 10% to about 20% by weight of the pharmaceutical formulation; and the lubricant component comprises from about 0.1% to about 2% by weight of the pharmaceutical formulation.

In some further embodiments, the active pharmacological agent comprises from about 1.4% to about 3.6% by weight of the pharmaceutical formulation; the filler/diluent component comprises from about 75% to about 85% by weight of the pharmaceutical formulation; the surface modifying agent component comprises from about 0.2% to about 1% by weight of the pharmaceutical formulation; the disintegrant component comprises from about 0.1% to about 0.6% by weight of the pharmaceutical formulation; the optional second filler/diluent component, when present, comprises from about 12% to about 18% by weight of the pharmaceutical formulation; and the lubricant component comprises from about 0.1% to about 1% by weight of the pharmaceutical formulation.

In some further embodiments, the active pharmacological agent comprises from about 2% to about 3% by weight of the pharmaceutical formulation; the filler/diluent component comprises from about 78% to about 83% by weight of the pharmaceutical formulation; the surface modifying agent component comprises from about 0.6% to about 0.9% by weight of the pharmaceutical formulation; the disintegrant component comprises from about 0.2% to about 0.5% by weight of the pharmaceutical formulation; the optional second filler/diluent, when present, component comprises from about 12% to about 18% by weight of the pharmaceutical formulation; and the lubricant component comprises from about 0.3% to about 0.7% by weight of the pharmaceutical formulation.

In some further embodiments, the active pharmacological agent comprises from about 1% to about 10% by weight of the pharmaceutical formulation; the filler/diluent component comprises from about 65% to about 85% by weight of the pharmaceutical formulation; the surface modifying agent component comprises from about 0.1% to about 3% by weight of the pharmaceutical formulation; the disintegrant component comprises from about 1% to about 8% by weight of the pharmaceutical formulation; the optional second filler/diluent component, when present, comprises from about 10% to about 20% by weight of the pharmaceutical formulation; and the lubricant component comprises from about 0.1% to about 2% by weight of the pharmaceutical formulation.

In some further embodiments, the active pharmacological agent comprises from about 2% to about 6% by weight of the pharmaceutical formulation; the filler/diluent component comprises from about 70% to about 80% by weight of the pharmaceutical formulation; the surface modifying agent component comprises from about 0.1% to about 2% by weight of the pharmaceutical formulation; the disintegrant component comprises from about 2% to about 6% by weight of the pharmaceutical formulation; the optional second filler/diluent component, when present, comprises from about 12% to about 18% by weight of the pharmaceutical formulation; and the lubricant component comprises from about 0.1% to about 1% by weight of the pharmaceutical formulation.

In still further embodiments, the active pharmacological agent comprises from about 3% to about 5% by weight of the pharmaceutical formulation; the filler/diluent component comprises from about 73% to about 77% by weight of the pharmaceutical formulation; the surface modifying agent component comprises from about 0.8% to about 1.3% by weight of the pharmaceutical formulation; the disintegrant component comprises from about 3% to about 5% by weight of the pharmaceutical formulation; the optional second filler/diluent component, when present, comprises from about 12% to about 18% by weight of the pharmaceutical formulation; and the lubricant component comprises from about 0.3% to about 0.7% by weight of the pharmaceutical formulation.

In some embodiments, the active pharmacological agent comprises from about 20% to about 40% by weight of the pharmaceutical formulation; the filler/diluent component comprises from about 45% to about 60% by weight of the pharmaceutical formulation; the surface modifying agent component comprises from about 4% to about 14% by weight of the pharmaceutical formulation; the disintegrant component comprises from about 1% to about 8% by weight of the pharmaceutical formulation; and the lubricant component comprises from about 0.1% to about 2% by weight of the pharmaceutical formulation.

In further embodiments, the active pharmacological agent comprises from about 27% to about 38% by weight of the pharmaceutical formulation; the filler/diluent component comprises from about 50% to about 56% by weight of the pharmaceutical formulation; the surface modifying agent component comprises from about 6% to about 12% by weight of the pharmaceutical formulation; the disintegrant component comprises from about 2% to about 6% by weight of the pharmaceutical formulation; and the lubricant component comprises from about 0.1% to about 1% by weight of the pharmaceutical formulation.

In further embodiments, the active pharmacological agent comprises from about 32% to about 35% by weight of the pharmaceutical formulation; the filler/diluent component comprises from about 52% to about 55% by weight of the pharmaceutical formulation; the surface modifying agent component comprises from about 8% to about 10% by weight of the pharmaceutical formulation; the disintegrant component comprises from about 3% to about 5% by weight of the pharmaceutical formulation; and the lubricant component comprises from about 0.3% to about 0.7% by weight of the pharmaceutical formulation.

In further embodiments, the active pharmacological agent comprises from about 10% to about 24% by weight of the pharmaceutical formulation; the filler/diluent component comprises from about 50% to about 70% by weight of the pharmaceutical formulation; the surface modifying agent component comprises from about 1% to about 10% by weight of the pharmaceutical formulation; the disintegrant component comprises from about 1% to about 8% by weight of the pharmaceutical formulation; the optional second filler/diluent component, when present, comprises from about 10% to about 20% by weight of the pharmaceutical formulation; and the lubricant component comprises from about 0.1% to about 2% by weight of the pharmaceutical formulation.

In further embodiments, the active pharmacological agent comprises from about 13% to about 20% by weight of the pharmaceutical formulation; the filler/diluent component comprises from about 55% to about 65% by weight of the pharmaceutical formulation; the surface modifying agent component comprises from about 2% to about 6% by weight of the pharmaceutical formulation; the disintegrant component comprises from about 2% to about 6% by weight of the pharmaceutical formulation; the optional second filler/diluent component, when present, comprises from about 12% to about 18% by weight of the pharmaceutical formulation; and the lubricant component comprises from about 0.1% to about 1% by weight of the pharmaceutical formulation.

In yet further embodiments, the active pharmacological agent comprises from about 15% to about 18% by weight of the pharmaceutical formulation; the filler/diluent component comprises from about 57% to about 62% by weight of the pharmaceutical formulation; the surface modifying agent component comprises from about 4% to about 5% by weight of the pharmaceutical formulation; the disintegrant component comprises from about 3% to about 5% by weight of the pharmaceutical formulation; the optional second filler/diluent component, when present, comprises from about 12% to about 18% by weight of the pharmaceutical formulation; and the lubricant component comprises from about 0.3% to about 0.7% by weight of the pharmaceutical formulation.

In some of the foregoing embodiments, the formulation contains from about 1 mg to about 125 mg of active pharmacological agent, from about 1 mg to about 3 mg of active pharmacological agent, from about 3 mg to about 7 mg of active pharmacological agent, from about 20 mg to about 30 mg of active pharmacological agent, from about 70 mg to about 80 mg of active pharmacological agent, or from about 90 mg to about 110 mg of active pharmacological agent.

The present invention also provides processes for preparing a pharmaceutical formulation of the invention as described herein, comprising:

a) mixing the active ingredient, at a portion of the filler/diluent, and the disintegrant, to form a mixture thereof; and

b) granulating the mixture with an aqueous solution comprising the surfactant to form a granulated mixture. In some embodiments, the processes further include the step of blending the granulated mixture with one or more of additional filler/diluent, second filler/diluent/diluent, or lubricant.

### DETAILED DESCRIPTION

In some embodiments, the present invention provides pharmaceutical formulations comprising a pharmaceutically effective amount of an active pharmacological agent and a carrier or excipient system, the carrier or excipient system comprising:

a) a filler/diluent component comprising from about 40% to about 90% by weight of the pharmaceutical formulation;

b) a surface modifying agent component comprising from about 0.4% to about 15% by weight of the pharmaceutical formulation;

c) a disintegrant component from about 0.01% to about 10% by weight of the pharmaceutical formulation;

d) optionally, a second filler/diluent component comprising up to about 20% by weight of the pharmaceutical formulation; and

e) a lubricant component comprising from about 0.01% to about 5% by weight of the pharmaceutical formulation;

wherein the active pharmacological agent has the Formula I: 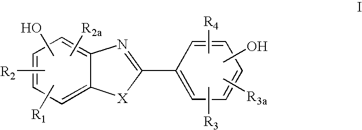
wherein

R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₅ and R₆ are each, independently hydrogen, alkyl of 1-6 carbon atoms, or aryl of 6-10 carbon atoms;

X is O, S, or N R₇; and

R₇ is hydrogen, alkyl of 1-6 carbon atoms or aryl of 6-10 carbon atoms, —COR₅, —CO₂R₅ or —SO₂R₅;

or a pharmaceutically acceptable salt thereof.

In some embodiments, X is O. In some further embodiments, R₁ is alkenyl of 2-3 carbon atoms, which is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆. In some embodiments, the active ingredient is 2-(3-fluoro-4-hydroxyphenyl)-7-vinyl-1,3-benzoxazol-5-ol (ERB-041) or a pharmaceutically acceptable salt thereof.

It will be understood that the weight percentages set forth for the filler/diluent component, surface modifying agent component, disintegrant component, optional second filler/diluent component, and lubricant component of the formulations disclosed herein are the percentages that each component will comprise of a final pharmaceutical formulation, without reference to any surface covering, such as a tablet coating or capsule. The remainder of the final formulation will be comprised of the active pharmacological agent(s).

Generally, the active pharmacological agent(s) can be present in from about 1.0% to about 50% by weight of the pharmaceutical formulation, from about 1.5% to about 40% by weight of the pharmaceutical formulation, or from about 2% to about 36% by weight of the pharmaceutical formulation. In some embodiments, the active pharmacological agent comprises 2-(3-fluoro-4-hydroxyphenyl)-7-vinyl-1,3-benzoxazol-5-ol or a pharmaceutically acceptable salt thereof.

In some embodiments, the filler/diluent component comprises from about 40% to about 90% by weight of the pharmaceutical formulation, from about 45% to about 85% by weight of the pharmaceutical formulation, or from about 50% to about 85% by weight of the pharmaceutical formulation. In some embodiments the optional second filler/diluent component is present and comprises from about 1% to about 20% of the pharmaceutical formulation. In some embodiments, the filler/diluent component and/or the optional second filler/diluent component, when present, include one or more agent that is useful as a filler or diluent or a combination of such agents. One or more fillers and/or one or more diluents may be selected in each case. In some embodiments, the filler/diluent component comprises a combination of mannitol and microcrystalline cellulose, and the optional second filler/diluent, when present, comprises mannitol and microcrystalline cellulose. Examples of such pharmaceutically acceptable fillers and/or diluent (and/or binding) agents include sugar or carbohydrate containing compounds such as mannitol, lactose, sucrose, powdered cellulose, microcrystalline cellulose, maltodextrin, sorbitol, starch, xylitol, and metal aluminosilicates such as magnesium aluminometasilicate (Neusilin®), as well as metal phosphates and carbonates. Other suitable filler/diluent materials can be found in, for example, Remington's Pharmaceutical Sciences, 17th ed., Mack Publishing Company, Easton, Pa., 1985, which is incorporated herein by reference in its entirety.

In some embodiments, the surface modifying agent component comprises from about 0.4% to about 15% by weight of the pharmaceutical formulation, from about 0.5 to about 12% by weight of the pharmaceutical formulation or from about 0.6 to about 10% by weight of the pharmaceutical formulation. The surface modifying agent can be any of the pharmaceutically acceptable wetting agents known in the art, for example, surfactants. Examples of such surface modifying agents include Poloxamer 188, sodium lauryl sulfate, polyoxyethylene sorbitan fatty acid esters, polyethylene glycols, polyoxyethylene castor oil derivatives, docusate sodium, quaternary ammonium amine compounds, sugar esters of fatty acids and glycerides of fatty acids. In some embodiments, the surface modifying agent component comprises one or more of Poloxamer 188 or sodium lauryl sulfate; preferably Poloxamer 188.

In some embodiments the disintegrant comprises from about 0.01% to about 10% by weight of the pharmaceutical formulation, from about 0.15% to about 8% by weight or the pharmaceutical formulation or from about 0.2% to about 6% of the pharmaceutical formulation. The disintegrant component can include one or more of the pharmaceutically acceptable agents known to be useful as a disintegrant. Examples of such include crosscarmellose sodium, pregelatinized starch, sodium starch glycolate, crospovidone, starch, alginic acid, sodium alginate, clay, cellulose floc, ion exchange resin, and effervescent systems based on food acids and an alkaline carbonate component

In some embodiments the lubricant comprises from about 0.01% to about 5.0% of the pharmaceutical formulation, from about 0.1% to about 2.0% of the pharmaceutical formulation, from about 0.1% to about 1.0% of the pharmaceutical formulation or from about 0.3% to about 0.7% of the pharmaceutical formulation. The lubricant can be selected from the many lubricants useful in the pharmaceutical arts. Examples of suitable lubricants include metallic stearates such as magnesium stearate, calcium stearate and zinc stearate, fatty acid esters, fatty acids, fatty alcohols, glyceryl behenate, mineral oil, paraffins, hydrogenated vegetable oils, leucine, polyethylene glycols, metallic lauryl sulfates, silica such as Aerosil® 200, and sodium chloride.

In some embodiments, processes are provided for the preparation of formulations described herein. In some embodiments, the processes comprise:

a) mixing the active ingredient, at a portion of the filler/diluent, and the disintegrant, to form a mixture thereof; and

b) spray granulating the mixture with an aqueous solution comprising the surfactant to form a granulated mixture.

In some embodiments, the processes further comprise the step of (c) blending the granulated mixture with one or more of additional filler/diluent, second filler/diluent/diluent, a lubricant, additional disintegrant, or additional surface modifiying agent. In some embodiments, the filler/diluent component comprises mannitol; the surface modifying agent component comprises Poloxamer 188; the disintegrant component comprises crosscarmellose sodium; the optional second filler/diluent component comprises microcrystalline cellulose; and the lubricant component comprises magnesium stearate.

In some further preferred embodiments, the formulation contains from about 1 mg to about 125 mg, or from about 1 mg to about 3 mg, or from about 3 mg to about 7 mg, or from about 20 mg to about 30 mg, or from about 70 mg to about 80 mg, or from about 90 mg to about 110 mg, of active pharmacological agent.

Oral formulations containing the present solid dispersions can comprise any conventionally used oral forms, including tablets, capsules, buccal forms, troches, lozenges and oral liquids, suspensions, and the like. Capsules or tablets containing the present solid dispersion can also be combined with mixtures of other active compounds or inert fillers and/or diluents such as pharmaceutically acceptable starches (e.g., corn, potato or tapioca starch), sugars, artificial sweetening agents, powdered celluloses such as crystalline and microcrystalline celluloses, flours, gelatins, gums, etc. In some embodiments, the formulations are solid dispersions contained in capsules, preferably spray granule dispersals in capsules.

Tablet formulations can be made by conventional compression, wet granulation, or dry granulation methods and utilize pharmaceutically acceptable fillers/diluents, binding agents, lubricants, disintegrants, suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Oral formulations used herein may utilize standard delay or time release formulations or spansules. Suppository formulations may be made from traditional materials, including cocoa butter, with or without the addition of waxes to alter the suppositories melting point, and glycerin. Water soluble suppository bases such as polyethylene glycols of various molecular weights may also be used.

Film coatings useful with the present formulations are known in the art and generally consist of a polymer (usually a cellulosic type of polymer), a colorant and a plasticizer. Additional ingredients such as wetting agents, sugars, flavors, oils and lubricants can be included in film coating formulations to impart certain characteristics to the film coat. The compositions and formulations herein may also be combined and processed as a solid, then placed in a capsule form such as a gelatin capsule.

The filler/diluent can comprise any substance known in the art that is useful for the preparation of solid oral formulations. Pharmaceutically acceptable fillers/diluents can be selected from any filler and/or diluent, for example, lactose, microcrystalline cellulose, sucrose, mannitol, calcium phosphate, calcium carbonate, powdered cellulose, maltodextrin, sorbitol, starch, xylitol, metal aluminosilicate such as magnesium aluminometasilicate (Neusilin®), those described above, and the like.

The present formulations can also include disintegrant agents. These disintegrants can be selected from those known in the art, including pregelatinized starch, sodium starch glycolate and the like. Other useful disintegrants include croscarmellose sodium, crospovidone, starch, alginic acid, sodium alginate, clay (e.g., veegum or xanthan gum), cellulose floc, ion exchange resin, or effervescent systems such as those utilizing food acids (such as citric acid, tartaric acid, malic acid, fumaric acid, lactic acid, adipic acid, ascorbic acid, aspartic acid, erythorbic acid, glutamic acid, and succinic acid) and an alkaline carbonate component (such as sodium bicarbonate, calcium carbonate, magnesium carbonate, potassium carbonate, ammonium carbonate, etc.). The disintegrant(s) useful herein can comprise from about 0.1% to about 10% of the formulation by weight, from about 0.15% to about 8%, or from about 0.2% to about 6%.

As will be appreciated, some components of the formulations of the invention can possess multiple functions. For example, a given component can act as both a diluent/filler and a disintegrant. In some such cases, the function of a given component can be considered singular, even though its properties may allow multiple functionality.

The pharmaceutical formulations and excipient systems herein can also contain an antioxidant or a mixture of antioxidants such as ascorbic acid. Other antioxidants that can be used include sodium ascorbate and ascorbyl palmitate, optionally in conjunction with an amount of ascorbic acid. An example range for the antioxidant(s) is from about 0.05% to about 15% by weight, from about 0.5% to about 15% by weight, or from about 0.5% to about 5% by weight. In some embodiments, the pharmaceutical formulations contain substantially no antioxidant.

Additional numerous various excipients, dosage forms, dispersing agents and the like that are suitable for use in connection with the solid dispersions of the invention are known in the art and described in, for example, Remington's Pharmaceutical Sciences, 17th ed., Mack Publishing Company, Easton, Pa., 1985, which is incorporated herein by reference in its entirety.

The materials, methods, and examples presented herein are intended to be illustrative, and are not intended to limit the scope of the invention.

### EXAMPLES

The following Examples illustrate preparation of solid dosage formulations of the present invention. The preparation of the solid dosage formulations, in some embodiments, involve initial preparation of a granulation comprising the active pharmacological agent. This entails first combining the active pharmacological agent with a portion of a filler/diluent and a portion of a glidant/disintegrant to form a mixture and then adding this mixture to an aqueous solution of including a portion of a surface modifying agent to form a final mixture that is dried, sieved and blended to form granules containing the active pharmacological agent. The granulation can be used to prepare solid dosage forms, e.g., capsules, of the present invention.

In some embodiments, the preparation of the solid dosage forms can further include blending the granules containing the active agent with one or more additional component such as additional filler/diluent, a second filler/diluent/diluent, a lubricant, additional disintegrant, or additional surface modifiying agent. The resulting mixture can be filled into capsules to the desired fill weight.

In some such embodiments, the portion of filler/diluent from the granulation containing the active agent comprises from about 0.1 to about 100%, about 5 to about 95%, about 10 to about 90%, about 15 to about 85%, about 20 to about 80%, about 25 to about 75%, about 30 to about 70%, about 35 to about 65%, about 40 to about 60%, about 45 to about 55%, about 50% of the total filler/diluent in the final capsule composition, i.e., not including any additional first filler/diluent or optional “second filler/diluent/diluent” as the term is used herein. In some embodiments, the portion of filler/diluent from the granulation containing the active agent comprises from about 60%, 61%, 62%, 63%, 64%, 65%, 66%, 67%, 68%, 69%, 70%, 71%, 72%, 73%, 74%, 75%, 76%, 77%, 78%, 79%, or 80% of the total filler/diluent in the final capsule composition. In some such embodiments, the additional filler/diluent comprises from about 0.1 to about 100%, about 5 to about 95%, about 10 to about 90%, about 15 to about 85%, about 20 to about 80%, about 25 to about 75%, about 30 to about 70%, about 35 to about 65%, about 40 to about 60%, about 45 to about 55%, about 50% of the total filler/diluent in the final capsule composition. In some embodiments, the additional filler/diluent comprises from about 20%, 21%, 22%, 23%, 24%, 25%, 26%, 27%, 28%, 29%, 30%, 31%, 32%, 33%, 34%, 35%, 36%, 37%, 38%, 39%, or 40% of the total filler/diluent in the final capsule composition. For example, one non-limiting embodiment, as illustrated in Example 6, the portion of filler/diluent (mannitol) from the granule prepared in Example 1 comprises about 66% of the filler/diluent in the final capsule composition, while the mannitol added later in the process comprises about 34% of the filler/diluent in the final capsule composition. Alternatively stated, in this example, a portion of about two-thirds of the filler/diluent used to make the capsules is used to prepare the granules containing the active agent to which the remaining one-third of the filler/diluent is added during final formulation preparation.

In some such embodiments, the portion of surface modifying agent from the granulation containing the active agent comprises from about 0.1 to about 100%, about 5 to about 95%, about 10 to about 90%, about 15 to about 85%, about 20 to about 80%, about 25 to about 75%, about 30 to about 70%, about 35 to about 65%, about 40 to about 60%, about 45 to about 55%, about 50% of the total surface modifying agent in the final capsule composition, i.e., not including any additional surface modifying agent. In some embodiments, the portion of surface modifying agent from the granulation containing the active agent comprises from about 60%, 61%, 62%, 63%, 64%, 65%, 66%, 67%, 68%, 69%, 70%, 71%, 72%, 73%, 74%, 75%, 76%, 77%, 78%, 79%, or 80% of the total surface modifying agent in the final capsule composition. In some such embodiments, the additional surface modifying agent comprises from about 0.1 to about 100%, about 5 to about 95%, about 10 to about 90%, about 15 to about 85%, about 20 to about 80%, about 25 to about 75%, about 30 to about 70%, about 35 to about 65%, about 40 to about 60%, about 45 to about 55%, about 50% of the total surface modifying agent in the final capsule composition. In some embodiments, the additional surface modifying agent comprises from about 20%, 21%, 22%, 23%, 24%, 25%, 26%, 27%, 28%, 29%, 30%, 31%, 32%, 33%, 34%, 35%, 36%, 37%, 38%, 39%, or 40% of the total surface modifying agent in the final capsule composition.

In some such embodiments, the portion of disintegrant from the granulation containing the active agent comprises from about 0.1 to about 100%, about 5 to about 95%, about 10 to about 90%, about 15 to about 85%, about 20 to about 80%, about 25 to about 75%, about 30 to about 70%, about 35 to about 65%, about 40 to about 60%, about 45 to about 55%, about 50% of the total disintegrant in the final capsule composition, i.e., not including the any additional disintegrant. In some embodiments, the portion of disintegrant from the granulation containing the active agent comprises from about 30%, 31%, 32%, 33%, 34%, 35%, 36%, 37%, 38%, 39%, 40%, 41%, 42%, 43%, 44%, 45%, 46%, 47%, 48%, 49%, or 50% of the total disintegrant in the final capsule composition. In some such embodiments, the additional disintegrant comprises from about 0.1 to about 100%, about 5 to about 95%, about 10 to about 90%, about 15 to about 85%, about 20 to about 80%, about 25 to about 75%, about 30 to about 70%, about 35 to about 65%, about 40 to about 60%, about 45 to about 55%, about 50% of the total disintegrant in the final capsule composition. In some embodiments, the additional disintegrant comprises from about 50%, 51%, 52%, 53%, 54%, 55%, 56%, 57%, 58%, 59%, 60%, 61%, 62%, 63%, 64%, 65%, 66%, 67%, 368%, 69%, or 70% of the total disintegrant in the final capsule composition

### Example 1

### Procedure for Preparation of ERB-041 Granule

Batch size=2.0-2.5 Kg.

1. Weigh out individually the Mannitol (Pearlitol® 200SD) USP, Croscarmellose Sodium EP/NF, Poloxamer 188 NF, and the ERB-041 (Micronised).
2. Weigh Purified Water into a suitable sized stainless steel beaker to make a 30.5% w/w solution of Poloxamer 188 NF.
3. Add the water to a suitable high shear granulator and then add the Poloxamer 188 NF. Start the impeller of the granulator at a low speed (chopper off) and continue to mix for a minimum of 45 minutes or until the Poloxamer is completely dissolved.
4. Combine and sieve the ERB-041 (Micronised), Mannitol (Pearlitol® 200SD) and Croscarmellose Sodium EP/NF through a 500 micron screen and transfer into the high shear granulator containing the Poloxamer Solution of Step 3.
5. Start the high shear granulator using the impeller set at an appropriate speed and the chopper set at an appropriate speed. The mixer may be stopped and the bowl scrapped, as required.
6. Continue processing until a suitable granule is produced adding additional water using an appropriate syringe, as necessary.
7. Pass the granule through an appropriate screen to de-agglomerate any large lumps and spread evenly on to an appropriate number of drying trays. Spread the oversize on a separate tray.
8. Dry the granules produced in an oven with a set-point of 50° C. for a minimum of 20 hours.
9. Combine the granules and sieve through an 800 micron screen.
10. Mill the greater than 800 micron size fraction granule with an appropriate screen, and sieve the milled material through the 800 micron screen, until a sufficiently small portion is retained.

11. Recombine the sieved materials from steps 9 and 10, and blend in a suitable mixer for 5 minutes to yield the final granule.

The composition of the granule is shown in the table below.

| Ingredient | % WT/WT |
|---|---|
| ERB-041 Micronised^{a} | 33.501 |
| Mannitol USP (Pearlitol ® 200SD) | 53.434 |
| Poloxamer 188 NF | 9.045 |
| Croscarmellose Sodium EP/NF | 4.020 |
| Purified Water USP^{b} | qs |
| TOTAL | 100.00 |

^{a}Potency of ERB-041 adjusted against Mannitol (Pearlitol ® 200SD)
^{b}Used in process but does not appear in final product

### Example 2

### Procedure for Preparation of Capsules Containing 100 MG of ERB-041

1. The Granule from Example 1 is blended with Magnesium Stearate and mixed.

2. #0 HPMC Capsules are filled with the blend to a target fill weight of 300.00 mg.

The composition of the capsule is shown in the table below.

| | | Input | |
|---|---|---|---|
| | | Dosage Unit | |
| Ingredient | % WT/WT | Input | Unit |
| ERB-041 | 33.333 | 100.0 | mg |
| Mannitol USP (Pearlitol ® 200SD) | 53.167 | 159.5^{a} | mg |
| Poloxamer 188 NF | 9.000 | 27.0 | mg |
| Croscarmellose Sodium EP/NF | 4.000 | 12.0 | mg |
| Magnesium Stearate NF/EP | 0.500 | 1.5 | mg |
| (Vegetable Extract) | | | |
| Purified Water USP | Qs | qs^{b} | |
| Capsule #0 HPMC Opaque | Qs | 1 | capsule |
| Brown, 4P Quali-V (Shionogi | | | |
| Qualicaps, inc. (Whitsett, NC)) | | | |
| Total | 100 | 300.0 | mg |

^{a}Potency of ERB-041 adjusted against Mannitol (Pearlitol ® 200SD)
^{b}Used in process, but does not appear in the final product

### Example 3

### Procedure for Preparation of Capsules Containing 75 MG of ERB-041

Capsules are prepared in identical fashion to that described in Example 2, except that the #0 HPMC Capsules are filled with the blend to a target fill weight of 225.00 mg.

### Example 4

### Procedure for Preparation of Capsules Containing 25 MG of ERB-041

1. Mill the Granule of Example 1 using an appropriate mill fitted with an appropriate screen and sieve through a 800 micron screen.
2. Sieve the Microcrystalline Cellulose NF (Avicel® PH200) through a 500 micron screen.
3. Blend the sieved material from Steps 1 and 2.
4. Sieve the additional Mannitol (Pearlitol® 200SD) and Croscarmellose Sodium EP/NF through a 500 micron screen and blend with material from Step 3.
5. Blend the material from step 4 with Magnesium Stearate EP/NF and mix.
6. Fill #0 HPMC Capsules with the blend from step 5 to a target fill weight of 150 mg.

The composition of the capsules is shown in the table below.

| | | Input/ | |
|---|---|---|---|
| | | Dosage Unit | |
| Ingredient | % WT/WT | Input | Unit |
| ERB-041 | 16.6667 | 25.00 | mg |
| Mannitol USP (Pearlitol 200SD) | 59.3333 | 89.00^{a} | mg |
| (Roquette America, Inc. (Keokuk, IA)) | | | |
| Poloxamer 188 NF | 4.5000 | 6.75 | mg |
| Croscarmellose Sodium EP/NF | 4.0000 | 6.00 | mg |
| Microcrystalline Cellulose NF (Avicel | 15.0000 | 22.50 | mg |
| PH200) | | | |
| Magnesium Stearate (Vegetable | 0.5000 | 0.75 | mg |
| Extract) NF/EP | | | |
| Purified Water USP | qs | qs^{b} | |
| Capsule #0 HPMC Opaque Brown, 4P | qs | 1 | capsule |
| Quali-V (Shionogi) | | | |
| Total | 100 | 150.00 | mg |

^{a}Potency of ERB-041 adjusted against Mannitol (Pearlitol ® 200SD)
^{b}Used in process, but does not appear in the final product

### Example 5

### Procedure for Preparation of Capsules Containing 5 MG of ERB-041

1. Mill the Granule of Example 1 using an appropriate mill fitted with an appropriate screen and sieve through an 800 micron screen.
2. Sieve the Microcrystalline Cellulose NF (Avicel® PH200) through a 500 micron screen.
3. Blend sieved material from Steps 1 and 2.
4. Sieve the additional Mannitol (Pearlitol® 200SD) and Croscarmellose Sodium EP/NF through a 500 micron screen and blend with material from Step 3.
5. Blend the material from Step 4 with Magnesium Stearate EP/NF and mix.
6. Fill #0 HPMC Capsules with the blend from Step 5 to a target fill weight of 124 mg.

The composition of the capsules is shown in the table below.

| | | Input/ | |
|---|---|---|---|
| | | Dosage Unit | |
| Ingredient | % WT/WT | Input | Unit |
| ERB-041 | 4.0323 | 5.00 | mg |
| Mannitol USP (Pearlitol ® 200SD) | 75.379 | 93.47^{a} | mg |
| (Roquette) | | | |
| Poloxamer 188 NF | 1.0887 | 1.35 | mg |
| Croscarmellose Sodium EP/NF | 4.000 | 4.96 | mg |
| Microcrystalline Cellulose NF | 15.0000 | 18.60 | mg |
| (Avicel ® PH200) | | | |
| Magnesium Stearate (Vegetable | 0.5000 | 0.62 | mg |
| Extract) NF/EP | | | |
| Purified Water USP | qs | | qs^{b} |
| Capsule #0 HPMC Opaque Brown, 4P | qs | 1 | capsule |
| Quali-V (Shionogi) | | | |
| TOTAL | 100 | 124.00 | mg |

^{a}Potency of ERB-041 adjusted against Mannitol (Pearlitol ® 200SD)
^{b}Used in process, but does not appear in the final product

### Example 6

### Procedure for Preparation of Capsules Containing 2 MG of ERB-041

1. Mill the Granule of Example 1 using an appropriate mill fitted with an appropriate screen and sieve through an 800 micron screen.
2. Sieve the Microcrystalline Cellulose NF (Avicel® PH200) through a 500 micron screen.
3. Blend sieved material from Steps 1 and 2.
4. Sieve the additional Mannitol (Pearlitol® 200SD) and Croscarmellose Sodium EP/NF through a 500 micron screen and blend with material from Step 3.
5. Blend the material from Step 4 with Magnesium Stearate EP/NF and mix.
6. Fill #0 HPMC Capsules with the blend from Step 5 to a target fill weight of 75 mg.

The composition of the capsules is shown in the table below.

| | | Input/ | |
|---|---|---|---|
| | | Dosage Unit | |
| Ingredient | % WT/WT | Input | Unit |
| ERB-041 | 2.667 | 2.000 | mg |
| Mannitol USP (Pearlitol 200SD) | 80.793 | 60.595^{a} | mg |
| (Roquette) | | | |
| Poloxamer 188 NF | 0.720 | 0.540 | mg |
| Croscarmellose Sodium EP/NF | 0.320 | 0.240 | mg |
| Microcrystalline Cellulose NF (Avicel | 15.000 | 11.250 | mg |
| PH200) | | | |
| Magnesium Stearate (Vegetable | 0.500 | 0.375 | mg |
| Extract) NF/EP | | | |
| Purified Water USP | qs | qs^{b} | |
| Capsule #0 HPMC Opaque Brown, | qs | 1 | capsule |
| 4P Quali-V (Shionogi) | | | |
| Total | 100 | 75.000 | mg |

^{a}Potency of ERB-041 adjusted against Mannitol (Pearlitol ® 200SD)
^{b}Not present in final product

### Example 7

### Procedure for Preparation of Capsules Containing ERB-041

A final granulation blend containing ERB-041 can be prepared as described in, for example, Example 5 and Example 6, except that additional disintegrant and/or additional surface modifying agent is/are added, for example, in Step 4. The additional disintegrant and/or additional surface modifying agent can be added along with additional filler/diluent, second filler/diluent/diluent and/or lubricant, or not.

It is intended that each of the patents, applications, and printed publications, including books, mentioned in this patent document be hereby incorporated by reference in their entirety.

As those skilled in the art will appreciate, numerous changes and modifications may be made to the embodiments of the invention without departing from the spirit of the invention. It is intended that all such variations fall within the scope of the invention.

What is claimed is:

1. A pharmaceutical formulation comprising a pharmaceutically effective amount of an active pharmacological agent and a carrier or excipient system, the carrier or excipient system comprising:
a) a filler/diluent component comprising from about 40% to about 90% by weight of the pharmaceutical formulation;
b) a surface modifying agent component comprising from about 0.4% to about 15% by weight of the pharmaceutical formulation;
c) a disintegrant component from about 0.01% to about 10% by weight of the pharmaceutical formulation;
d) optionally, a second filler/diluent component comprising up to about 20% by weight of the pharmaceutical formulation; and
e) a lubricant component comprising from about 0.01% to about 5% by weight of the pharmaceutical formulation;
wherein the active pharmacological agent has the Formula I: 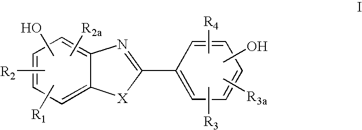
wherein
R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently hydrogen, alkyl of 1-6 carbon atoms, or aryl of 6-10 carbon atoms;
X is O, S, or N R₇; and
R₇ is hydrogen, alkyl of 1-6 carbon atoms or aryl of 6-10 carbon atoms, —COR₅, —CO₂R₅ or —SO₂R₅;
or a pharmaceutically acceptable salt thereof.

2. The pharmaceutical formulation of claim 1 wherein X is O.

3. The pharmaceutical formulation of claim 2, wherein R₁ is alkenyl of 2-3 carbon atoms, which is optionally substituted with hydroxyl, —CN, halogen, trifluroalkyl, trifluoroalkoxy, —COR₅, —CO₂R₅, —NO₂, CONR₅R₆, NR₅R₆ or N(R₅)COR₆.

4. The pharmaceutical formulation of claim 1 wherein the active ingredient is 2-(3-fluoro-4-hydroxyphenyl)-7-vinyl-1,3-benzoxazol-5-ol or a pharmaceutically acceptable salt thereof.

5. The pharmaceutical formulation of claim 1 or claim 4 wherein the active pharmacological agent comprises from about 1.0% to about 50% by weight of the pharmaceutical formulation.

6. The pharmaceutical formulation of claim 1 or claim 4 wherein:
the active pharmacological agent comprises from about 1.0% to about 50% by weight of the pharmaceutical formulation;
the filler/diluent component comprises from about 40% to about 90% by weight of the pharmaceutical formulation;
the surface modifying agent component comprises from about 0.4% to about 15% by weight of the pharmaceutical formulation;
the disintegrant component comprises from about 0.1% to about 10% by weight of the pharmaceutical formulation;
the second filler/diluent component, when present, comprises up to about 20% by weight of the pharmaceutical formulation; and
the lubricant component comprises from about 0.01% to about 5% by weight of the pharmaceutical formulation.

7. The pharmaceutical formulation of claim 1 or claim 4 wherein:
the active pharmacological agent comprises from about 1.5% to about 40% by weight of the pharmaceutical formulation;
the filler/diluent component comprises from about 45% to about 85% by weight of the pharmaceutical formulation;
the surface modifying agent component comprises from about 0.5 to about 12% by weight of the pharmaceutical formulation;
the disintegrant component comprises from about 0.15% to about 8% by weight of the pharmaceutical formulation;
the second filler/diluent component, when present, comprises from about 1% to about 20% by weight of the pharmaceutical formulation; and
the lubricant component comprises from about 0.1% to about 2% by weight of the pharmaceutical formulation.

8. The pharmaceutical formulation of claim 1 or claim 4 wherein:
the active pharmacological agent comprises from about 2% to about 36% by weight of the pharmaceutical formulation;
the filler/diluent component comprises from about 50% to about 85% by weight of the pharmaceutical formulation;
the surface modifying agent component comprises from about 0.6 to about 10% by weight of the pharmaceutical formulation;
the disintegrant component comprises from about 0.2% to about 6% by weight of the pharmaceutical formulation;
the second filler/diluent component, when present, comprises from about 1.0% to about 20% by weight of the pharmaceutical formulation; and
the lubricant component comprises from about 0.1% to about 1% by weight of the pharmaceutical formulation.

9. The pharmaceutical formulation of claim 1 or claim 4 wherein the filler/diluent component comprises one or more of mannitol, lactose, sucrose, powdered cellulose, microcrystalline cellulose, maltodextrin, sorbitol, starch, xylitol, metal aluminosilicate or magnesium aluminometasilicate.

10. The pharmaceutical formulation of claim 1 or claim 4 wherein the filler/diluent component comprises mannitol.

11. The pharmaceutical formulation of claim 1 or claim 4 wherein the surface modifying agent component comprises a surfactant.

12. The pharmaceutical formulation of claim 1 or claim 4 wherein the surface modifying agent component comprises one or more of Poloxamer 188, sodium lauryl sulfate, polyoxyethylene sorbitan fatty acid ester, polyethylene glycol, polyoxyethylene castor oil derivative, docusate sodium, quaternary ammonium amine compound, sugar esters of fatty acid, or glycerides of fatty acid.

13. The pharmaceutical formulation of claim 1 or claim 4 wherein the surface modifying agent component comprises one or more of Poloxamer 188 or sodium lauryl sulfate.

14. The pharmaceutical formulation of claim 1 or claim 4 wherein the surface modifying agent component comprises Poloxamer 188.

15. The pharmaceutical formulation of claim 1 or claim 4 wherein the disintegrant component comprises one or more of crosscarmellose sodium, pregelatinized starch, sodium starch glycolate, crospovidone, starch, alginic acid, sodium alginate, clay, cellulose floc, ion exchange resin, or effervescent systems based on food acids and an alkaline carbonate component.

16. The pharmaceutical formulation of claim 1 or claim 4 wherein the disintegrant component comprises crosscarmellose sodium.

17. The pharmaceutical formulation of claim 1 or claim 4 wherein the optional second filler/diluent component comprises one or more of microcrystalline cellulose, mannitol, lactose, sucrose, powdered cellulose, maltodextrin, sorbitol, starch, xylitol, metal aluminosilicate, or magnesium aluminometasilicate.

18. The pharmaceutical formulation of claim 1 of claim 4 wherein the optional second filler/diluent component comprises microcrystalline cellulose.

19. The pharmaceutical formulation of claim 1 or claim 4 wherein the lubricant component comprises one or more of metallic stearate, fatty acid ester, fatty acid, fatty alcohol, glyceryl behenate, mineral oil, parrafin, hydrogenated vegetable oil, leucine, polyethylene glycol, metallic lauryl sulfate, or sodium chloride.

20. The pharmaceutical formulation of claim 19 wherein the metallic stearate is magnesium stearate, calcium stearate or zinc stearate.

21. The pharmaceutical formulation of claim 1 or claim 4 wherein the lubricant component comprises magnesium stearate.

22. The pharmaceutical formulation of claim 1 wherein:
the filler/diluent component comprises one or more of mannitol, lactose, sucrose, powdered cellulose, microcrystalline cellulose, maltodextrin, sorbitol, starch, xylitol, metal aluminosilicate, or magnesium aluminometasilicate;
the surface modifying agent component comprises one or more of Poloxamer 188, sodium lauryl sulfate, polyoxyethylene sorbitan fatty acid ester, polyethylene glycol, polyoxyethylene castor oil derivative, docusate sodium, quaternary ammonium amine compound, sugar esters of fatty acid, or glycerides of fatty acids;
the disintegrant component comprises one or more of crosscarmellose sodium, pregelatinized starch, sodium starch glycolate, crospovidone, starch, alginic acid, sodium alginate, clay, cellulose floc, ion exchange resin, or effervescent systems based on food acids and an alkaline carbonate component;
the disintegrant component comprises crosscarmellose sodium;
the optional second filler/diluent component comprises one or more of microcrystalline cellulose, mannitol, lactose, sucrose, powdered cellulose, maltodextrin, sorbitol, starch, xylitol, metal aluminosilicate, or magnesium aluminometasilcate; and
the lubricant component comprises one or more of metallic stearate, fatty acid esters, fatty acid, fatty alcohol, glyceryl behenate, mineral oil, parrafin, hydrogenated vegetable oil, leucine, polyethylene glycol, metallic lauryl sulfate, or sodium chloride.

23. The pharmaceutical formulation of claim 4 wherein:
the filler/diluent component comprises one or more of mannitol, lactose, sucrose, powdered cellulose, microcrystalline cellulose, maltodextrin, sorbitol, starch, xylitol, metal aluminosilicate, or magnesium aluminometasilcate;
the surface modifying agent component comprises one or more of Poloxamer 188, sodium lauryl sulfate, polyoxyethylene sorbitan fatty acid ester, polyethylene glycol, polyoxyethylene castor oil derivative, docusate sodium, quaternary ammonium amine compound, sugar esters of fatty acid, or glycerides of fatty acid;
the disintegrant component comprises one or more of crosscarmellose sodium, pregelatinized starch, sodium starch glycolate, crospovidone, starch, alginic acid, sodium alginate, clay, cellulose floc, ion exchange resin, or effervescent systems based on food acids and an alkaline carbonate component;
the disintegrant component comprises crosscarmellose sodium;
the optional second filler/diluent component comprises one or more of microcrystalline cellulose, mannitol, lactose, sucrose, powdered cellulose, maltodextrin, sorbitol, starch, xylitol, metal aluminosilicate, or magnesium aluminometasilcate; and
the lubricant component comprises one or more of metallic stearate, fatty acid ester, fatty acid, fatty alcohol, glyceryl behenate, mineral oil, parrafin, hydrogenated vegetable oil, leucine, polyethylene glycol, metallic lauryl sulfate, or sodium chloride.

24. The pharmaceutical formulation of claim 1 wherein:
the filler/diluent component comprises mannitol;
the surface modifying agent component comprises Poloxamer 188;
the disintegrant component comprises crosscarmellose sodium;
the optional second filler/diluent component comprises microcrystalline cellulose; and
the lubricant component comprises magnesium stearate.

25. The pharmaceutical formulation of claim 4 wherein:
the filler/diluent component comprises mannitol;
the surface modifying agent component comprises Poloxamer 188;
the disintegrant component comprises crosscarmellose sodium;
the optional second filler/diluent component comprises microcrystalline cellulose; and
the lubricant component comprises magnesium stearate.

26. The pharmaceutical formulation of any of claims 1, 4 and 22-25 wherein:
the active pharmacological agent comprises from about 1.0% to about 5% by weight of the pharmaceutical formulation;
the filler/diluent component comprises from about 70% to about 90% by weight of the pharmaceutical formulation;
the surface modifying agent component comprises from about 0.1% to about 2% by weight of the pharmaceutical formulation;
the disintegrant component comprises from about 0.01% to about 2% by weight of the pharmaceutical formulation;
the second filler/diluent component comprises from about 10% to about 20% by weight of the pharmaceutical formulation; and
the lubricant component comprises from about 0.1% to about 2% by weight of the pharmaceutical formulation.

27. The pharmaceutical formulation of any of claims 1, 4 and 22-25 wherein:
the active pharmacological agent comprises from about 1.4% to about 3.6% by weight of the pharmaceutical formulation;
the filler/diluent component comprises from about 75% to about 85% by weight of the pharmaceutical formulation;
the surface modifying agent component comprises from about 0.2% to about 1% by weight of the pharmaceutical formulation;
the disintegrant component comprises from about 0.1% to about 0.6% by weight of the pharmaceutical formulation;
the second filler/diluent component comprises from about 12% to about 18% by weight of the pharmaceutical formulation; and
the lubricant component comprises from about 0.1% to about 1% by weight of the pharmaceutical formulation.

28. The pharmaceutical formulation of any of claims 1, 4 and 22-25 wherein:
the active pharmacological agent comprises from about 2% to about 3% by weight of the pharmaceutical formulation;
the filler/diluent component comprises from about 78% to about 83% by weight of the pharmaceutical formulation;
the surface modifying agent component comprises from about 0.6% to about 0.9% by weight of the pharmaceutical formulation;
the disintegrant component comprises from about 0.2% to about 0.5% by weight of the pharmaceutical formulation;
the second filler/diluent component comprises from about 12% to about 18% by weight of the pharmaceutical formulation; and
the lubricant component comprises from about 0.3% to about 0.7% by weight of the pharmaceutical formulation.

29. The pharmaceutical formulation of any of claims 1, 4 and 22-25 wherein:
the active pharmacological agent comprises from about 1% to about 10% by weight of the pharmaceutical formulation;
the filler/diluent component comprises from about 65% to about 85% by weight of the pharmaceutical formulation;
the surface modifying agent component comprises from about 0.1% to about 3% by weight of the pharmaceutical formulation;
the disintegrant component comprises from about 1% to about 8% by weight of the pharmaceutical formulation;
the second filler/diluent component comprises from about 10% to about 20% by weight of the pharmaceutical formulation; and
the lubricant component comprises from about 0.1% to about 2% by weight of the pharmaceutical formulation.

30. The pharmaceutical formulation of any of claims 1, 4 and 22-25 wherein:
the active pharmacological agent comprises from about 2% to about 6% by weight of the pharmaceutical formulation;
the filler/diluent component comprises from about 70% to about 80% by weight of the pharmaceutical formulation;
the surface modifying agent component comprises from about 0.1% to about 2% by weight of the pharmaceutical formulation;
the disintegrant component comprises from about 2% to about 6% by weight of the pharmaceutical formulation;
the second filler/diluent component comprises from about 12% to about 18% by weight of the pharmaceutical formulation; and
the lubricant component comprises from about 0.1% to about 1% by weight of the pharmaceutical formulation.

31. The pharmaceutical formulation of any of claims 1, 4 and 22-25 wherein:
the active pharmacological agent comprises from about 3% to about 5% by weight of the pharmaceutical formulation;
the filler/diluent component comprises from about 73% to about 77% by weight of the pharmaceutical formulation;
the surface modifying agent component comprises from about 0.8% to about 1.3% by weight of the pharmaceutical formulation;
the disintegrant component comprises from about 3% to about 5% by weight of the pharmaceutical formulation;
the second filler/diluent component comprises from about 12% to about 18% by weight of the pharmaceutical formulation; and
the lubricant component comprises from about 0.3% to about 0.7% by weight of the pharmaceutical formulation.

32. The pharmaceutical formulation of any of claims 1, 4 and 22-25 wherein:
the active pharmacological agent comprises from about 20% to about 40% by weight of the pharmaceutical formulation;
the filler/diluent component comprises from about 45% to about 60% by weight of the pharmaceutical formulation;
the surface modifying agent component comprises from about 4% to about 14% by weight of the pharmaceutical formulation;
the disintegrant component comprises from about 1% to about 8% by weight of the pharmaceutical formulation; and
the lubricant component comprises from about 0.1% to about 2% by weight of the pharmaceutical formulation.

33. The pharmaceutical formulation of any of claims 1, 4 and 22-25 wherein:
the active pharmacological agent comprises from about 27% to about 38% by weight of the pharmaceutical formulation;
the filler/diluent component comprises from about 50% to about 56% by weight of the pharmaceutical formulation;
the surface modifying agent component comprises from about 6% to about 12% by weight of the pharmaceutical formulation;
the disintegrant component comprises from about 2% to about 6% by weight of the pharmaceutical formulation; and
the lubricant component comprises from about 0.1% to about 1% by weight of the pharmaceutical formulation.

34. The pharmaceutical formulation of any of claims 1, 4 and 22-25 wherein:
the active pharmacological agent comprises from about 32% to about 35% by weight of the pharmaceutical formulation;
the filler/diluent component comprises from about 52% to about 55% by weight of the pharmaceutical formulation;
the surface modifying agent component comprises from about 8% to about 10% by weight of the pharmaceutical formulation;
the disintegrant component comprises from about 3% to about 5% by weight of the pharmaceutical formulation; and
the lubricant component comprises from about 0.3% to about 0.7% by weight of the pharmaceutical formulation.

35. The pharmaceutical formulation of any of claims 1, 4 and 22-25 wherein:
the active pharmacological agent comprises from about 10% to about 24% by weight of the pharmaceutical formulation;
the filler/diluent component comprises from about 50% to about 70% by weight of the pharmaceutical formulation;
the surface modifying agent component comprises from about 1% to about 10% by weight of the pharmaceutical formulation;
the disintegrant component comprises from about 1% to about 8% by weight of the pharmaceutical formulation;
the second filler/diluent component comprises from about 10% to about 20% by weight of the pharmaceutical formulation; and
the lubricant component comprises from about 0.1% to about 2% by weight of the pharmaceutical formulation.

36. The pharmaceutical formulation of any of claims 1, 4 and 22-25 wherein:
the active pharmacological agent comprises from about 13% to about 20% by weight of the pharmaceutical formulation;
the filler/diluent component comprises from about 55% to about 65% by weight of the pharmaceutical formulation;
the surface modifying agent component comprises from about 2% to about 6% by weight of the pharmaceutical formulation;
the disintegrant component comprises from about 2% to about 6% by weight of the pharmaceutical formulation;
the second filler/diluent component comprises from about 12% to about 18% by weight of the pharmaceutical formulation; and
the lubricant component comprises from about 0.1% to about 1% by weight of the pharmaceutical formulation.

37. The pharmaceutical formulation of any of claims 1, 4 and 22-25 wherein:
the active pharmacological agent comprises from about 15% to about 18% by weight of the pharmaceutical formulation;
the filler/diluent component comprises from about 57% to about 62% by weight of the pharmaceutical formulation;
the surface modifying agent component comprises from about 4% to about 5% by weight of the pharmaceutical formulation;
the disintegrant component comprises from about 3% to about 5% by weight of the pharmaceutical formulation;
the second filler/diluent component comprises from about 12% to about 18% by weight of the pharmaceutical formulation; and
the lubricant component comprises from about 0.3% to about 0.7% by weight of the pharmaceutical formulation.

38. The pharmaceutical formulation of claim 1 wherein the formulation contains from about 1 mg to about 125 mg of active pharmacological agent.

39. The pharmaceutical formulation of claim 1 wherein the formulation contains from about 1 mg to about 3 mg of active pharmacological agent.

40. The pharmaceutical formulation of claim 1 wherein the formulation contains from about 3 mg to about 7 mg of active pharmacological agent.

41. The pharmaceutical formulation of claim 1 wherein the formulation contains from about 20 mg to about 30 mg of active pharmacological agent.

42. The pharmaceutical formulation of claim 1 wherein the formulation contains from about 70 mg to about 80 mg of active pharmacological agent.

43. The pharmaceutical formulation of claim 1 wherein the formulation contains from about 90 mg to about 110 mg of active pharmacological agent.

44. The pharmaceutical formulation of claim 4 wherein the formulation contains from about 1 mg to about 125 mg of active pharmacological agent.

45. The pharmaceutical formulation of claim 4 wherein the formulation contains from about 1 mg to about 3 mg of active pharmacological agent.

46. The pharmaceutical formulation of claim 4 wherein the formulation contains from about 3 mg to about 7 mg of active pharmacological agent.

47. The pharmaceutical formulation of claim 4 wherein the formulation contains from about 20 mg to about 30 mg of active pharmacological agent.

48. The pharmaceutical formulation of claim 4 wherein the formulation contains from about 70 mg to about 80 mg of active pharmacological agent.

49. The pharmaceutical formulation of claim 4 wherein the formulation contains from about 90 mg to about 110 mg of active pharmacological agent.

50. The pharmaceutical formulation of claim 26 wherein the formulation contains from about 1 mg to about 3 mg of active pharmacological agent.

51. The pharmaceutical formulation of claim 27 wherein the formulation contains from about 1 mg to about 3 mg of active pharmacological agent.

52. The pharmaceutical formulation of claim 28 wherein the formulation contains from about 1 mg to about 3 mg of active pharmacological agent.

53. The pharmaceutical formulation of claim 29 wherein the formulation contains from about 3 mg to about 7 mg of active pharmacological agent.

54. The pharmaceutical formulation of claim 30 wherein the formulation contains from about 3 mg to about 7 mg of active pharmacological agent.

55. The pharmaceutical formulation of claim 31 wherein the formulation contains from about 3 mg to about 7 mg of active pharmacological agent.

56. The pharmaceutical formulation of claim 32 wherein the formulation contains from about 20 mg to about 30 mg of active pharmacological agent.

57. The pharmaceutical formulation of claim 33 wherein the formulation contains from about 20 mg to about 30 mg of active pharmacological agent.

58. The pharmaceutical formulation of claim 34 wherein the formulation contains from about 20 mg to about 30 mg of active pharmacological agent.

59. The pharmaceutical formulation of claim 32 wherein the formulation contains from about 70 mg to about 80 mg of active pharmacological agent.

60. The pharmaceutical formulation of claim 33 wherein the formulation contains from about 70 mg to about 80 mg of active pharmacological agent.

61. The pharmaceutical formulation of claim 34 wherein the formulation contains from about 70 mg to about 80 mg of active pharmacological agent.

62. The pharmaceutical formulation of claim 32 wherein the formulation contains from about 90 mg to about 110 mg of active pharmacological agent.

63. The pharmaceutical formulation of claim 33 wherein the formulation contains from about 90 mg to about 110 mg of active pharmacological agent.

64. The pharmaceutical formulation of claim 34 wherein the formulation contains from about 90 mg to about 110 mg of active pharmacological agent.

65. A process for preparing a pharmaceutical formulation of claim 1 or claim 4, the process comprising:
a) mixing the active ingredient, at least a portion of the filler/diluent, and at least a portion of the disintegrant, to form a mixture thereof; and
b) granulating the mixture with an aqueous solution comprising at least a portion of the surface modifying agent component to form a granulated mixture.

66. A process for preparing a pharmaceutical formulation of claim 1 or claim 4, the process comprising:
a) mixing the active ingredient, at least a portion of the filler/diluent, and at least a portion of the disintegrant, to form a mixture thereof; and
b) spray granulating the mixture with an aqueous solution comprising at least a portion of the surface modifying agent component to form a granulated mixture.

67. The process of claim 65 further comprising the step of blending the granulated mixture with one or more of additional filler/diluent, second filler/diluent/diluent, lubricant, additional disintegrant, or additional surface modifying agent.

68. The process of claim 66 further comprising the step of blending the granulated mixture with one or more of additional filler/diluent, second filler/diluent/diluent, lubricant, additional disintegrant, or additional surface modifying agent.

69. The process of claim 65 wherein:
the filler/diluent component comprises one or more of mannitol, lactose, sucrose, powdered cellulose, microcrystalline cellulose, maltodextrin, sorbitol, starch, xylitol, metal aluminosilicate, or magnesium aluminometasilicate;
the surface modifying agent component comprises one or more of Poloxamer 188, sodium lauryl sulfate, polyoxyethylene sorbitan fatty acid ester, polyethylene glycol, polyoxyethylene castor oil derivative, docusate sodium, quaternary ammonium amine compound, sugar esters of fatty acid, or glycerides of fatty acids;
the disintegrant component comprises one or more of crosscarmellose sodium, pregelatinized starch, sodium starch glycolate, crospovidone, starch, alginic acid, sodium alginate, clay, cellulose floc, ion exchange resin, or effervescent systems based on food acids and an alkaline carbonate component;
the disintegrant component comprises crosscarmellose sodium;
the optional second filler/diluent component comprises one or more of microcrystalline cellulose, mannitol, lactose, sucrose, powdered cellulose, maltodextrin, sorbitol, starch, xylitol, metal aluminosilicate, or magnesium aluminometasilicate; and
the lubricant component comprises one or more of metallic stearate, fatty acid ester, fatty acid, fatty alcohol, glyceryl behenate, mineral oil, parrafin, hydrogenated vegetable oil, leucine, polyethylene glycol, metallic lauryl sulfate, or sodium chloride.

70. The process of claim 65 wherein:
the filler/diluent component comprises mannitol;
the surface modifying agent component comprises Poloxamer 188;
the disintegrant component comprises crosscarmellose sodium;
the optional second filler/diluent component comprises microcrystalline cellulose; and
the lubricant component comprises magnesium stearate.

71. The process of claim 70 wherein the formulation contains from about 1 mg to about 125 mg of active pharmacological agent.

72. The process of claim 70 wherein the formulation contains from about 1 mg to about 3 mg of active pharmacological agent.

73. The process of claim 70 wherein the formulation contains from about 3 mg to about 7 mg of active pharmacological agent.

74. The process of claim 70 wherein the formulation contains from about 20 mg to about 30 mg of active pharmacological agent.

75. The process of claim 70 wherein the formulation contains from about 70 mg to about 80 mg of active pharmacological agent.

76. The process of claim 70 wherein the formulation contains from about 90 mg to about 110 mg of active pharmacological agent.

77. A product of the process of claim 65.

78. A product of the process of claim 66.

79. A product of the process of claim 67.

80. A product of the process of claim 69.

81. A product of the process of claim 70.

82. A product of the process of claim 71.

83. A capsule or tablet comprising a pharmaceutical formulation of claim 1.
